(19) United States
(12) Patent Application Publication
(10) Pub. No.: US 2008/0091249 A1
(43) Pub. Date: Apr. 17, 2008

(54) Photobiomodulation Apparatus with Enhanced Performance and Safety Features

(75) Inventor: Sean Xiaolu Wang, Wilmington, DE (US)

Correspondence Address: BWT PROPERTY, INC., 19 SHEA WAY, SUITE 301, NEWARK, DE, 19713, US

(73) Assignee: BWT PROPERTY, INC., Newark, DE (US)

(21) Appl. No.: 11/869,222

(22) Filed: Oct. 9, 2007

Related U.S. Application Data
(60) Provisional application No. 60/828,982, filed on Oct. 11, 2006.

(51) Int. Cl. A61B 18/18

(52) U.S. Cl. 607/88

(57) ABSTRACT

A photobiomodulation apparatus providing precise light intensity, light dosage, and tissue temperature control so as to enhance the safety of the photobiomodulation treatment process and improve the comfort level of the patient.

### REFERENCE TO RELATED APPLICATIONS

This application claims an invention which was disclosed in Provisional Patent Application No. 60/828,982, filed Oct. 11, 2006, entitled “Photobiomodulation Apparatus with Enhanced Performance and Safety Features.” The benefit under 35 USC §119(e) of the above mentioned United States Provisional Applications is hereby claimed, and the aforementioned applications are hereby incorporated herein by reference.

### FIELD OF THE INVENTION

The present invention relates generally to a photobiomodulation apparatus and more specifically to a photobiomodulation apparatus with enhanced performance and safety features.

### BACKGROUND

Photobiomodulation or photobiostimulation relates to treatment of living tissue with certain wavelength of light to aid tissue regeneration, resolve inflammation, relieve pain, and boost the immune system. Clinical applications include soft tissue injuries, chronic pain, wound healing, nerve regeneration, and possibly even resolving viral and bacterial infections.

Photobiomodulation is generally performed with a laser light source. Depending on the area of the treatment site, the power of the laser may range from several milliwatts to tens of watts. The involvement of high power lasers place a safety issue as high light intensity may cause overheating, denaturizing, or even carbonization of the tissue. Here light intensity is defined as the total laser power divided by the area of the treatment site. For photobiomodulation applications, where the treatment site is relatively large, it is actually the light intensity that sets the tissue damage threshold.

In PCT patent application No. WO 01/78830, Casey et al. discloses a photobiomodulation treatment apparatus that incorporates a thermo-graphic device, such as an infrared camera to detect infrared radiation emitted by the targeted tissue and produce a thermograph. The thermograph is used to control the laser output energy to impart precisely controlled light dosage to the targeted tissue. The Casey patent application fails to teach a method for light intensity control.

In U.S. patent application No. 2004/0162596, Altshuler et al. discloses a method for modulating the efficacy of photobiomodulation by controlling the temperature in the targeted region and/or its surrounding volume. The method comprises the steps of measuring the temperature of the targeted region and modifying the heat delivered to or extracted from the targeted region to keep its temperature within a pre-defined threshold. The method does not comprise any step for light intensity control.

In U.S. Pat. No. 6,475,211, Chess et al. discloses a method and apparatus for treatment of biologic tissue with simultaneous radiation and temperature modification. The temperature modification, which is performed by a vortex tube, helps to reduce pain and other side effects caused by the light radiation. The Chess patent does not provide any clue for controlling the intensity of the radiation light source.

There thus exists a need in the art for a photobiomodulation apparatus with precise light intensity, dosage, and tissue temperature control so as to enhance the performance as well as safety of the treatment process and improve the comfort level of the patient.

### SUMMARY OF THE INVENTION

According to one aspect of the present invention, there is provided a plurality of sensor elements in the photobiomodulation apparatus to monitor the treatment process. Such sensor elements include photo detectors to monitor the power of the lasers, distance measurement devices to monitor the distance between the laser output port and the treatment site, as well as remote temperature sensors to monitor the temperature of the treatment site.

According to another aspect of the present invention, there is provided a temperature modulation unit in the photobiomodulation apparatus to control the temperature of the targeted tissue during the treatment process.

According to yet another aspect of the present invention, there is provided at least two laser units in the photobiomodulation apparatus. The two laser units have different output powers and beam divergence angles to treat targeted tissue with different areas. Yet in another possible configuration, the two laser units have different output wavelengths, resulting in different absorption coefficient and penetration depth in the targeted tissue. The light dosage at different depth of the tissue can thus be controlled by controlling the light intensity of each laser unit.

According to yet another aspect of the present invention, there is provided a control unit in the photobiomodulation apparatus. The control unit can respond to the sensor signal produced by the sensor elements, control the status of the laser units and the temperature modulation unit, as well as send alarm signal to the operator of the photobiomodulation apparatus in case the light intensity or the tissue temperature exceeds a pre-defined range.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates one exemplary embodiment of the photobiomodulation apparatus.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

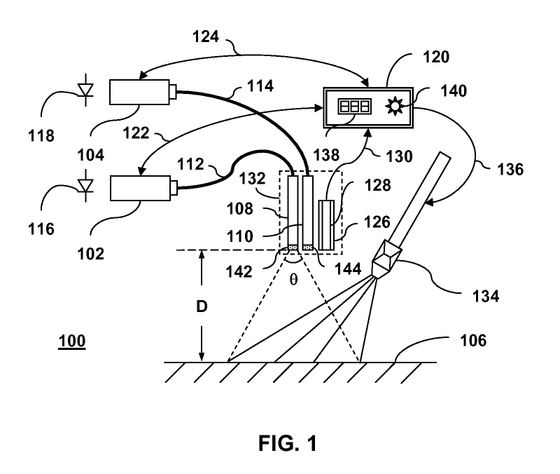

FIG. 1 illustrates one exemplary embodiment of the present invention. The photobiomodulation apparatus 100 comprises two laser units 102 and 104. The laser unit 102 has a relatively high output power level of several watts to several tens of watts. The laser unit 104 has a relatively low output power level of several milliwatts to several hundreds of milliwatts. The types of the lasers used may include but are not limited to diode lasers, fiber lasers, solid state lasers, and gas lasers. The output wavelength of the laser units may range from ultraviolet, visible to near infrared or even mid-infrared. The light of the two laser units 102 and 104 is delivered to the targeted tissue 106 through individual output wands 108 and 110, respectively. The wands 108 and 110 may have different numerical apertures for laser beam divergence angle control. For example, the wand 108 associated with the high power laser unit 102 may have a relatively larger numerical aperture so that the corresponding laser beam have a larger divergence angle (θ) to cover a large-area treatment site. Meanwhile, the wand 110 associated with the low power laser unit 104 may have a relatively smaller numerical aperture so that the corresponding laser beam can be utilized to treat small-area tissue. This double-laser design avoids the safety problem when a high power laser is used to treat a small-area target, in which case the light intensity of the laser beam has a chance to exceed the safety level. The two laser units 102 and 104 are connected with their output wands 108 and 110 through optical fibers (or other forms of optical waveguides) 112 and 114, respectively. In case where the two output wands 108 and 110 are designed as detachable elements, a wand identification mechanism such as those disclosed by Kelsoe et al. in U.S. Pat. No. 5,085,492 may be introduced to prevent wand misconnection. In this exemplary embodiment, two photo detectors 116 and 118 are used to measure the output power (P) of the corresponding laser units 102 and 104 and the measured power level is sent to a central control unit 120 through electrical connections 122 and 124, respectively. The central control unit 120 can control the on/off status, drive current (or power level) of the two laser units 102 and 104 through the same electrical connections 122 and 124.

The photobiomodulation apparatus 100 further comprises a distance measurement unit 126 and a remote temperature sensor 128. The distance measurement unit 126 can be a simple caliper, or more preferably a laser or ultrasound distance measurement device, which measures the distance (D) between the output port of the wand 108 and 110 to the targeted tissue 106. The measured distance data are sent to the central control unit 120 through an electrical connection 130. The size (A) of the laser beam on the targeted tissue can be calculated as:

A=π·(D·tan (θ/2))̂2

where D is the measured distance value, and θ is the divergence angle of the laser beam set by the numerical aperture of the output wand 108 and 110. Thus the light intensity (I) of the laser beam can be determined as:

I=P/A

where P is the output power of the laser units 102 and 104 measured by the photo detectors 116 and 118. The obtained light intensity can be displayed to the operator by a display unit 138 on the central control unit 120. The light dosage, which is a product of the light intensity (I) and the duration time (T) of treatment process, can be automatically controlled by the central control unit 120 or be manually controlled by the operator. In case the light intensity exceeds a safety level or is beyond a predefined optimum range for photobiomodulation, the central control unit 120 may send a warning signal to the operator through an indicator 140. The operator can thus correct the light intensity by adjusting the power of the laser units 102, 104 and/or the distance between the wand 108, 110 and the targeted tissue 106. When the light intensity exceeds above a pre-defined safety level, the central control unit 120 may automatically shut down the laser units 102 and 104.

The remote temperature sensor 128 is preferably an infrared thermometer, which is capable of measuring the average tissue temperature for the treatment site. The accuracy for the temperature sensor 128 is preferably better than 1 degree Celsius (° C.). The measured temperature data are also sent to the central control unit 120 through the electrical connection 130. When the tissue temperature exceeds a pre-defined range, a warning message is generated by the indicator 140. The central control unit 120 may shut down the laser units 102 and 104 in case the tissue temperature is too high. In this exemplary embodiment, the output wands 108, 110, the distance measurement unit 126, and the temperature sensor 128 may be integrated together to form a common output port 132 for ease of operation. To further enhance the uniformity of the laser beam, optical diffusers 142, 144 may be attached in front of the output wands 108, 110 to homogenize the laser beam.

The photobiomodulation apparatus 100 further comprises a temperature modulation unit 134 to control the temperature of the targeted tissue 106. The temperature modulation unit 134 can be a dynamic cooling device as disclosed by Nelson et al. in U.S. Pat. No. 5,814,040 or a vortex tube as disclosed by Chess et al. in U.S. Pat. No. 6,475,211, both are hereby incorporated by reference. When a high intensity laser is used in the photobiomodulation process to produce high penetration depth into the tissue, the surface temperature of the tissue may exceed a safety level due to excessive heat generation. In this case, the temperature modulation unit 134 may deliver cold material to the treatment site to keep the tissue temperature below the safety level. The central control unit 120 can control the heat extraction rate of the temperature modulation unit 134 through an electrical connection 136 based on the measured light intensity on the tissue 106 and the tissue temperature measured by the remote temperature sensor 128. In another case, the temperature control unit 134 may also deliver warm material to the treatment site to modulate the efficacy of photobiomodulation.

In a slight variation of the present embodiment, the photobiomodulation apparatus comprises a plurality of laser units with different output wavelengths. The light of the plurality of laser units may be applied simultaneously or alternatively on the targeted tissue. Since the absorption rate and penetration depth of the laser light is mainly determined by its wavelength, the light dosage at different depth of the tissue can thus be controlled by controlling the light intensity of each laser unit. For example, the laser light with high penetration depth and low penetration depth may be applied alternatively or be mixed in certain ratio on the target tissue so that more even treatment effects can be obtained for different depth of the tissue than in the case where only one laser wavelength is used. As another advantage, the multiple-wavelength operation mode avoids the heat accumulation problem at a specific depth of the tissue where the light absorption rate has the maximum value at one laser wavelength.

In another variation of the present embodiment, the output power of the laser units may be modulated to produce a pulsed light output. The light intensity of the laser units can thus be controlled by varying the duty cycle of the power modulation to keep the average light intensity as well as the temperature of the targeted tissue below a safety threshold.

In yet another variation of the present embodiment, the photobiomodulation apparatus further comprises another photo detector to monitor the radiation emitted by the tissue in case it is carbonized by the laser beam. The central control unit may shut down the laser units when such a radiation is detected to protect the targeted tissue.

In the foregoing specification, specific embodiments of the present invention have been described. However, one of ordinary skill in the art appreciates that various modifications and changes can be made without departing from the scope of the present invention as set forth in the claims below. For example, the laser units in the disclosed photobiomodulation apparatus may be replaced by light emitting diodes (LEDs). Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. An apparatus for performing photobiomodulation on a targeted tissue, the apparatus comprising:
at least one light source to produce light emission from an output port to the targeted tissue, wherein said light emission has a divergence angle set by the properties of said light source and output port;
at least one photo detector to measure the optical power of said light emission;
a distance sensor to measure the distance between the output port and the targeted tissue;
a temperature sensor to monitor the temperature of the targeted tissue;
a temperature modulation unit to control the temperature of the targeted tissue; and
a central control unit to control the status of said light source and temperature modulation unit based on the information obtained from said photo detector, distance sensor, and temperature sensor.

2. The apparatus of claim 1, wherein the central control unit measures the light intensity on the targeted tissue based on the divergence angle and optical power of the light emission along with the distance between the output port and the targeted tissue.

3. The apparatus of claim 2, wherein the central control unit controls a drive current of the light source to keep the measured light intensity within a pre-defined range.

4. The apparatus of claim 2, wherein the light source is modulated to produce a light intensity modulation, and wherein the central control unit controls a duty cycle of said intensity modulation to keep the measured average light intensity within a pre-defined range.

5. The apparatus of claim 1, wherein the central control unit controls the temperature modulation unit to keep the tissue temperature within a pre-defined range.

6. The apparatus of claim 2, wherein the central control unit sends alarm signal to an operator when the measured light intensity and/or the tissue temperature exceed a pre-defined range.

7. The apparatus of claim 2, wherein the central control unit automatically shut down the light source when the measured light intensity and/or the tissue temperature are greater than a pre-defined safety level.

8. The apparatus of claim 1, wherein the light sources comprise at least two laser units, and wherein the optical power of the two laser units can be adjusted independently during the photobiomodulation process.

9. The apparatus of claim 8, wherein one laser unit has a relatively higher optical power to treat large-area tissue and the other laser unit has a relatively smaller optical power to treat small-area tissue.

10. The apparatus of claim 8, wherein the two laser units have different output wavelengths to treat tissue at different depth.

11. The apparatus of claim 1, further comprising a photo detector to monitor the radiation emitted by the targeted tissue in case it is carbonized by the light emission produced by the light source, and wherein the central control unit automatically shut down the light source when said radiation is detected.

12. A method for performing photobiomodulation on a targeted tissue, the method comprising the steps of:
providing at least one light source to produce light emission;
delivering said light emission from an output port to the targeted tissue;
monitoring the light intensity on the targeted tissue by measuring the optical power of the light emission and the distance between the output port and the targeted tissue;
controlling the light intensity on the targeted tissue to keep it within a pre-defined range;
providing a temperature sensor to monitor the temperature of the targeted tissue;
controlling the temperature of the targeted tissue to keep it within a pre-defined range.
